(19) United States
(12) Patent Application Publication
(10) Pub. No.: US 2015/0344552 A1
(43) Pub. Date: Dec. 3, 2015

(54) ANTIBODY AGAINST THE PROTEIN TRIO AND ITS METHOD OF PRODUCTION

(71) Applicants: CENTRE NATIONAL DE LA RECHERCHE SCIENTIFIQUE, Paris-Cedex (FR); UNIVERSITE MONTPELLIER 2 SCIENCES ET TECHNIQUES, Montpellier Cedex 5 (FR)

(72) Inventors: Anne DEBANT, PRADES-LE-LEZ (FR); Jerome BOUDEAU, MONTPELLIER (FR)

(73) Assignees: UNIVERSITE MONTPELLIER 2 SCIENCES ET TECHNIQUES, Montpellier Cedex 5 (FR); CENTRE NATIONAL DE LA RECHERCHE SCIENTIFIQUE, Paris Cedex 16 (FR)

(21) Appl. No.: 14/650,068

(22) PCT Filed: Dec. 6, 2013
(86) PCT No.: PCT/EP2013/075865

§ 371 Date: Jun. 5, 2015

(30) Foreign Application Priority Data
Dec. 7, 2012 (EP) 12306543.5

(51) Int. Cl. C07K 16/18; G01N 33/574; C07K 14/47

(52) U.S. Cl. CPC C07K 16/18; C07K 14/47; G01N 33/57496; G01N 2800/52; G01N 2333/47

(57) ABSTRACT

Disclosed are specific antibodies against the protein Trio, their method of production, and their use in in vitro cancer prognostic method. Also described are the antigens enabling production of the antibodies.

The present invention relates to specific antibodies against the protein Trio, their method of production, and their use in in vitro cancer prognostic method. The present invention also relates to the antigens enabling to produce said antibodies.

Rho GTPases are involved in numerous processes, such as proliferation, transformation, cell migration and morphogenesis, by their ability to direct the remodeling of the actin cytoskeleton in different cellular systems (Etienne-Manneville, et Hall, (2002) Nature, 420: 629-635). These proteins are, also involved in different oncogenic processes, in particular in metastatic progression by the activation of GTPases Rac1 and RhoA (Karlsson et al., (2009)Biochim Biophys Acta. 1762(2):91-98).

The protein Trio is a member of the family of Rho-GEFs (guanine nucleotide-exchange factors) (Debant et al., (1996) Proc. Natl. Acad. Sci. U.S.A. 93, 5466-5471), which positively regulate the activation of Rho GTPases by promoting the exchange of GDP for GTP. Trio is a 340-kDa protein that contains two GEF domains (GEFD1 and GEFD2) and a serine kinase domain. The first Rho-GEF domain (TrioGEFD1) activates RhoG (Blangy et al., (2000) J Cell Sci 113, 729-739), which in turn activates Rac1 and Cdc42, and promotes anchorage-independent cell growth (Seipel et al., (1999) J Cell Sci 112, 1825-1834), whereas the second Rho-GEF domain (TrioGEFD2) acts specifically on RhoA. In addition to these domains, Trio contains also numerous accessory domains, such as 8 spectrin repeats, two SH3 domains and an immunoglobulin-like domain (Debant et al., 1996), which participate in protein-protein interactions. Trio has been established as a master gene involved in cell motility, axon outgrowth and guidance, mainly through the activation of Rac1 by its first GEF domain. Besides full-length Trio, there are also 3 isoforms of Trio (TrioA, TrioC and TrioD), having one or two GEF domains and generated from alternative splicing (Portales-Casamar et al., (2006) Biol. Cell 98:183-193). These isoforms are specifically expressed in the nervous system, at a higher level than the full-length Trio, which is ubiquitously expressed.

Recent studies showing over-expression of Trio in different tumoral tissues suggest that Trio could also be involved in oncogenesis (Gibault et al., (2011) J. Pathol. 223(1):64-71; Lane et al., (2008) Int Semin Surg Oncol 5:23; Zheng et al., (2004) Am J Pathol 165:63-69; Salhia et al. (2008) Am J Pathol 173:1828-1838). In addition, one oncogenic isoform of Trio called Tgat has been isolated from ATL (adult-T cell leukemia) patients. (Yoshizuka, et al J Biol Chem, (2004) 279:43998-4004)

Based on the data obtained by CGH-array (comparative genomic hybridization) and transcriptome analysis of soft tissue sarcoma with complex genetic profile and bad prognosis,—the study of Gibault et al. (2011) has suggested that the gene trio, located at Chromosome Arm 5p, could be a genetic marker of a subtype of soft-tissue cancers that have poor prognostic.

Lane et al. have also observed a high expression level of Trio in breast tumours, especially in those with poor prognosis.

A study by Perou et al. ((2000) Nature 406: 747-752) classified breast cancers according to molecular subtypes: luminal A (LA), luminal B (LB), triple-negative (TN), HER2-enriched and normal breast-like tumors. However, about half of the HER2 overexpressing tumors are ER-positive and fall into the LB subtype, the normal breast-like group seems to be an artifact, and ER-negative tumors encompass at least three other molecular subgroups: the molecular apocrine (MA) breast tumor group, the interferon-rich subgroup and the claudin-low subtype.

Consequently, it is necessary to perform prognosis analysis by integrating clinicopathological criteria, microenvironmental parameters and selected immunohistochemical markers that can be detected by sensitive and specific antibodies.

The previous studies have suggested that Trio expression could be an important genetic marker for cancer diagnostic or prognosis. However, besides previously described nucleic acid probes (U.S. Pat. No. 5,994,070) designed for the detection of Trio expression at the mRNA level, so far there is no specific and sensitive antibody that could detect the over expression of Trio protein in human tissues.

In the past, the Inventors of the present invention have tried to generate antibodies against the TrioC-specific C-terminal end, but with no success (Portales-Casamar et al., (2006) Biol. Cell 98:183-193).

Recently some anti-Trio antibodies have been produced by using different fragments of Trio as antigens, but none of them presents the specificity and sensitivity to be used in immunocytochemistry, immunoprecipitation or immunohistochemistry.

Taking into account these elements, there is a great necessity to develop a sensitive and specific antibody against Trio.

The first aspect of the present invention is to provide a new antigen that could be used to produce said antibody.

Another aspect of the present invention is to provide a specific and sensitive antibody against Trio.

Another aspect of the present invention is also to provide an in vitro cancer prognostic method.

One of the aspects of the present invention is also to provide an in vitro prognostic method from soft tissue sarcoma with complex genomic profile and poor prognosis.

One of the aspects of the present invention is also to provide an in vitro breast cancer, in particular triple negative breast cancer, prognostic method.

According to a general embodiment, the antigen of the present invention is constituted of from 50 to 400 amino acids, said antigen comprising a polypeptide P1,
which is a peptide of at least 50 contiguous amino acids belonging to the polypeptide represented by sequence SEQ ID NO: 1; or
which is a peptide having at least 90%, in particular 95%, especially 98% of sequence homology with the amino acid sequence of SEQ ID NO: 1.

According to a general embodiment, the antigen of the present invention comprises or consists in from 50 to 400 amino acids, said antigen comprising a polypeptide P1,
which is a peptide of at least 50 contiguous amino acids belonging to the polypeptide represented by sequence SEQ ID NO: 1,
   with the proviso that

LSGGCELTVVIHDFTACNSNELTIRRGQTVEVLERPHDKPDWCLVRTTDR
SPAAEGLVPCGSLCIAHSRSSMEMEGIFNHK is excluded,

or
which is a peptide having at least 90%, or at least 91%, or at least 92%, or at least 93%, or at least 94%, in particular 95%, or at least 96%, or at least 97%, especially 98% or at least 99%, of sequence homology with the amino acid sequence of SEQ ID NO: 1,
   with the proviso that

LSGGCELTVVIHDFTACNSNELTIRRGQTVEVLERPHDKPDWCLVRTTDR
SPAAEGLVPCGSLCIAHSRSSMEMEGIFNHK is excluded.

The peptide excluded is a 81 amino acids peptide corresponding to accession number CQ128429, disclosed in WO0157272-A2/37288, Aug. 9, 2001 aeomica, Inc (US). This 81 amino acids peptide can be excluded from all pertinent embodiments.

According to another general embodiment, the antigen of the present invention is constituted of from 82 to 400 amino acids, said antigen comprising a polypeptide P1,
which is a peptide of at least 50 contiguous amino acids belonging to the polypeptide represented by sequence SEQ ID NO: 1; or
which is a peptide having at least 90%, or at least 91%, or at least 92%, or at least 93%, or at least 94%, in particular 95%, or at least 96%, or at least 97%, especially 98% or at least 99%, of sequence homology with the amino acid sequence of SEQ ID NO: 1.

In a particular embodiment the antigen of the present invention is constituted of a peptide sequence having at least 90%, or at least 91%, or at least 92%, or at least 93%, or at least 94%, in particular 95%, or at least 96%, or at least 97%, especially 98%, or at least 99%, of sequence homology with the antigen having from 82 to 400 amino acids, said antigen comprising a polypeptide P1 represented by sequence SEQ ID NO: 1.

According to a general embodiment, the antigen of the present invention is constituted of or consists in from 50 to 400 amino acids, said antigen comprising a polypeptide P1,
which is a peptide of at least 50 contiguous amino acids belonging to the polypeptide represented by sequence SEQ ID NO: 1; or
which is a peptide having at least 90%, or at least 91%, or at least 92%, or at least 93%, or at least 94%, in particular 95%, or at least 96%, or at least 97%, especially 98%, or at least 99%, of sequence homology with the amino acid sequence of SEQ ID NO: 1.

The present invention relies on the immunological experimental data of a fragment of the TrioC protein containing the SH3 domain of said protein, said fragment being surprisingly able to generate the production of a specific and sensitive anti-Trio antibody. In fact, the new antigen of the present invention, in contrast to all expectations, can enable the production of antibodies that can specifically recognize the protein Trio and isoforms TrioA, TrioC and TrioD.

The Inventors of the present invention show for the first time that the SH3 domain of TrioC is the key element of the antigen of the present invention. The presence of this domain or a functional fragment of this domain makes the antigen of the present invention capable of producing specific antibodies against the protein Trio and its isoforms containing this motif.

An antigen constituted of from 50 to 400 amino acids, said antigen comprising a polypeptide P1,
which is a peptide of at least 50 contiguous amino acids belonging to the polypeptide represented by sequence SEQ ID NO: 1;
or
which is a peptide having at least 90%, in particular 95%, especially 98% of sequence homology with the amino acid sequence of SEQ ID NO: 1,
in particular the polypeptide P1 being represented by amino acid sequence SEQ ID NO: 1, or any antigen according to the invention for its use in the treatment or the prophylaxis of cancer, is therefore part of the present invention.

The expression “antigen” in the context of the present invention refers to a polypeptide which can enable the production of one or more antibodies and could bind to said one or more antibodies, or fragment Fab, F(ab′)₂, SdAb (single-domain antibody) or scFv (single chain variable fragment) of said antibodies by it(s) epitope(s).

The antigen according to the present invention excludes the peptide having the following sequence:

LSGGCELTVVIHDFTACNSNELTIRRGQTVEVLERPHDKPDWCLVRTTDR
SPAAEGLVPCGSLCIAHSRSSMEMEGIFNHK.

The expression “antibody” refers to a protein containing two heavy chains and two light chains, each of them containing one or more constant regions and a variable region. The variable regions of a heavy chain and a light chain constitute the antigen binding site of said antibody.

“SdAb (single-domain antibody)” fragment refers to an antibody fragment consisting of a single monomeric variable antibody domain. Said variable domain can be a variable domain of a heavy chain or a light chain of an above defined antibody.

“ScFv (single chain variable fragment)” refers to a fusion protein of the variable regions of the heavy (VH) and light chains (VL) of an above defined antibody, connected with a short linker peptide.

Fab (fragment antigen-binding) fragment refers to a region of an antibody constituted of one constant and one variable domain of each of the heavy and the light chain of an above defined antibody.

F(ab′)₂ fragment is constituted of two Fab fragments linking by a disulphide bond.

The polypeptide represented by sequence SEQ ID NO: 1 corresponds to the SH3 domain of TrioC.

The sequence homology percentage can be calculated according to a conventional sequence alignment program, such as the mathematical algorithm described by Karlin and Altschul ((1993) Proc. Natl. Acad. Sci USA 90: 5873-5877). Such an algorithm is e.g. incorporated into the BLASTn and BLASTp programs of Altschul et al. ((1990) J. Mol. Biol. 215: 403-410), which are available at internet site of NCBI (http://blast.ncbi.nlm.nih.gov/Blast.cgi?PAGE=MegaBlast&PROGRAM=blastn&BLAST_PROGRAMS=megaBlast&PAGE_TYPE=BlastSearch&SHOW_DEFAULTS=on&BLAST_SPEC=blast2seq&QUERY=&SUBJECTS=).

The determination of the percent of identity is preferably performed with the standard parameters of the BLASTn and BLASTp programs.

In a particular embodiment, the antigen of the invention comprises or consists in a polypeptide P1 which is represented by amino acid sequence SEQ ID NO: 1.

In another particular embodiment, the antigen of the invention comprises or consists in a polypeptide P1, wherein the N-terminal of said polypeptide P1 is linked to a N-terminal flanking region, said N-terminal flanking region having at least 80%, in particular 95%, especially 98% of sequence homology with a fragment of a polypeptide having the amino acid sequence of SEQ ID NO: 8, said fragment being situated from position 1520 to position 1596 of the sequence SEQ ID NO: 8.

The sequence SEQ ID NO: 8 corresponds to the endogenous N-terminal flanking region of SH3 domain.

In a more particular embodiment, the antigen of the invention comprises or consists in a polypeptide P1, wherein the N-terminal flanking region is represented by amino acid sequence SEQ ID NO: 9.

In another particular embodiment the antigen of the invention comprises or consists in a polypeptide P1, wherein the N-terminal flanking region is represented by a sequence having at least 90%, at least 91%, at least 92% at least 93% at least 94% at least 95%, at least 96%, at least 97%, at least 98%, or at least 99% homology with amino acid sequence ID NO:9

In another particular embodiment, the antigen of the invention comprises or consists in a polypeptide P1, wherein the N-terminal of said polypeptide P1 is linked to a N-terminal flanking region, said N-terminal flanking region having less than 50%, in particular less than 40%, especially less than 20% of sequence homology with a fragment of a polypeptide having the amino acid sequence of SEQ ID NO: 8, wherein the N-terminal extremity of said fragment is located from position 1520 to position 1560 of the sequence SEQ ID NO: 8 and the C-terminal extremity of said fragment is located from position 1561 to position 1596 of the sequence SEQ ID NO: 8.

A more particular embodiment of the invention relates to an antigen comprising or consisting in:
a polypeptide P1, situated at N-terminal part of said antigen, having the amino acid sequence of SEQ ID NO: 1 or an amino acid sequence having at least 90%, in particular 95%, especially 98% of sequence homology with the sequence of SEQ ID NO: 1, and
a polypeptide P2, situated at C-terminal part of said antigen, having the amino acid sequence of SEQ ID NO: 2 or an amino acid sequence having at least 90%, in particular 95%, especially of 98% of sequence homology with the sequence of SEQ ID NO:2.

The N-terminal of said polypeptide P1 can be linked to a N-terminal flanking region, having at least 80%, in particular 95%, especially 98% of sequence homology with a fragment of a polypeptide having the amino acid sequence of SEQ ID NO: 8, said fragment being situated from position 1520 to position 1596 of the sequence SEQ ID NO: 8. Particularly, the N-terminal flanking region can be the peptide represented by SEQ ID NO: 9.

The N-terminal of said polypeptide P1 can also be linked to a N-terminal flanking region, having less than 50%, in particular less than 40%, especially less than 20% of sequence homology with a fragment of a polypeptide having the amino acid sequence of SEQ ID NO: 8, wherein the N-terminal extremity of said fragment is located from position 1520 to position 1560 of the sequence SEQ ID NO: 8 and the C-terminal extremity of said fragment is located from position 1561 to position 1596 of the sequence SEQ ID NO: 8.

The sequence SEQ ID NO: 2 corresponds to the specific TrioC C-terminal end.

In one embodiment, said antigen is having at least 85%, at least 87%, at least 88%, at least 89%, or at least 90% homology with a polypeptide P1, situated at N-terminal part of said antigen, having the amino acid sequence of SEQ ID NO: 1 and a polypeptide P2, situated at C-terminal part of said antigen, having the amino acid sequence of SEQ ID NO: 2.

In another particular embodiment, the antigen of the invention comprises or consists in a polypeptide P3 having at least from 100 to 300 amino acids between the C-terminal part of the polypeptide P1 and the N-terminal part of the polypeptide P2.

In still another embodiment, the antigen of the invention comprises or consists in a polypeptide P3 having at least from 100 to 300 amino acids between the C-terminal part of the polypeptide P1, having the amino acid sequence of SEQ ID NO: 1 and the N-terminal part of the polypeptide P2, having the amino acid sequence of SEQ ID NO: 2 or an amino acid sequence having at least 90%, in particular 95%, especially of 98% of sequence homology with the sequence of SEQ ID NO:2.

In still another embodiment, the antigen of the invention comprises or consists in a polypeptide P3 having at least from 100 to 300 amino acids between the C-terminal part of the polypeptide P1 and the N-terminal part of a polypeptide P2, having at least 91%, or at least 92%, or at least 93% or at least 94%, in particular 95%, or at least 96%, or at least 97%, especially of 98% or 99% of sequence homology with the sequence of SEQ ID NO:2.

In still another embodiment, the antigen of the invention comprises or consists in a polypeptide having at least from 100 to 300 amino acids between the C-terminal part of the polypeptide P1, having the amino acid sequence of SEQ ID NO: 1 or an amino acid sequence having at least 90%, in particular 95%, especially of 98% of sequence homology with the sequence of SEQ ID NO:1 and the N-terminal part of the polypeptide P2 having the amino acid sequence of SEQ ID NO: 2 or an amino acid sequence having at least 90%, in particular 95%, especially of 98% of sequence homology with the sequence of SEQ ID NO:2

In one advantageous embodiment of an antigen according to the aforementioned invention, said polypeptide P3 has amino acid sequence of SEQ ID NO: 3 or an amino acid sequence having at least 90%, in particular 95%, more in particular 98% of sequence homology with SEQ ID NO: 3.

In one embodiment, said antigen is having at least 85%, at least 87%, at least 88%, or at least 89%, homology with polypeptide P3.

The sequence SEQ ID NO: 3 corresponds to the endogenous peptide in the TrioC protein, which is located between the C-terminal extremity of the SH3 domain having the sequence of SEQ ID NO: 1 and the N-terminal extremity of the specific TrioC C-terminal end represented by the sequence SEQ ID NO: 2.

In another advantageous embodiment of an antigen according to the aforementioned invention, said polypeptide P3 is a fragment of from 50 to 165 contiguous amino acids of the peptide represented by SEQ ID NO: 3.

In another advantageous embodiment, said polypeptide P3 has the N-terminal extremity located at position 1 of the sequence SEQ ID: 3 and the C-terminal extremity located from position 50 to position 165 of the sequence SEQ ID NO: 3.

In a particularly advantageous embodiment, an antigen according to the present invention comprises or consists in:
a polypeptide P1, situated at N-terminal part of said antigen, having the amino acid sequence of SEQ ID NO: 1;
a polypeptide P2, situated at C-terminal part of said antigen, having the amino acid sequence of SEQ ID NO: 2; and
a polypeptide P3, between the C-terminal extremity of the polypeptide P1 and the N-terminal extremity of the polypeptide P2, having the amino acid sequence of SEQ ID NO: 3.

In another particularly advantageous embodiment, an antigen according to the present invention comprises or consists in an amino acid represented by the sequence of SEQ ID NO: 4 or an amino acid sequence having at least 90%, in particular 95%, more in particular 98% of sequence homology with SEQ ID NO:4.

More advantageously, an antigen of the present invention is represented by the amino acid sequence of SEQ ID NO: 4.

The Invention also relates to a nucleic acid comprising or consisting of a nucleic acid coding for the peptide sequence of the above defined antigen.

The invention excludes any nucleic acid coding for the peptide sequence herein excluded.

Particularly, the invention relates to a nucleic acid comprising or consisting in a nucleic acid coding for the polypeptide P1 represented by the amino acid sequence of SEQ ID NO: 1 or an amino acid sequence having at least 90%, in particular 95%, more in particular 98% sequence homology with the sequence of SEQ ID NO: 1.

In a particular embodiment, the invention concerns a nucleic acid comprising or consisting in:
a nucleic acid coding for the polypeptide P1 represented by the amino acid sequence of SEQ ID NO: 1 or an amino acid sequence having at least 90%, in particular 95%, more in particular 98% sequence homology with the sequence of SEQ ID NO: 1, and
another nucleic acid coding for the polypeptide P2 represented by the amino acid sequence of SEQ ID NO: 2 or an amino acid sequence having at least 90%, in particular 95%, more in particular 98% sequence homology with the sequence of SEQ ID NO:2.

In a more particular embodiment, the invention relates to a nucleic acid comprising or consisting in:
a nucleic acid N1 having the sequence of SEQ ID NO: 5 and coding for the polypeptide P1 represented by the sequence SEQ ID NO: 1, and
a nucleic acid N2 having the sequence of SEQ ID NO: 6 and coding for the polypeptide P2 represented by the sequence SEQ ID NO: 2

In another particular embodiment, the invention relates to a nucleic acid comprising or consisting in:
a nucleic acid coding for the polypeptide P1 represented by the amino acid sequence of SEQ ID NO: 1 or an amino acid sequence having at least 90%, in particular 95%, more in particular 98% sequence homology with the sequence of SEQ ID NO: 1, and
another nucleic acid coding for the polypeptide P2 represented by the amino acid sequence of SEQ ID NO: 2 or an amino acid sequence having at least 90%, in particular 95%, more in particular 98% sequence homology with the sequence of SEQ ID NO:2, and
a third nucleic acid coding for the polypeptide P3 represented by the amino acid sequence of SEQ ID NO: 3 or an amino acid sequence having at least 90%, in particular 95%, more in particular 98% sequence homology with the sequence of SEQ ID NO:3.

More particularly, the invention relates to a nucleic acid comprising or consisting in:
a nucleic acid N1 having the sequence of SEQ ID NO: 5 and coding for the polypeptide P1 represented by the sequence SEQ ID NO: 1,
a nucleic acid N2 having the sequence of SEQ ID NO: 6 and coding for the polypeptide P2 represented by the sequence SEQ ID NO: 2, and
a nucleic acid N3 having the sequence of SEQ ID NO: 10 and coding for the polypeptide P3 represented by the sequence SEQ ID NO: 3.

In an advantageous embodiment, the present invention concerns a nucleic acid having the sequence SEQ ID NO: 7, coding for the amino acid represented by the sequence of SEQ ID NO: 4.

The present invention also relates to a recombinant vector, especially a plasmid, a cosmid, a phage or a DNA virus, containing a nucleotide sequence as defined above.

More particularly, the present invention relates to a recombinant vector containing a nucleotide sequence SEQ ID NO: 7.

In one advantageous embodiment, the invention relates to the recombinant vector previously defined, containing the elements necessary for the expression in a host cell of the polypeptides, or peptide, coded by the nucleic acids above defined, inserted in said vector.

The invention also relates to a host cell, in particular chosen from bacteria, viruses, yeasts, fungi, plants or mammalian cells, the said host cell being transformed, especially by means of a vector defined above, in such a way that its genome contains a nucleotide sequence defined above.

The present invention provides also a specific and sensitive anti-Trio antibody.

Said antibody is produced by an aforementioned antigen and is directed against above defined antigen, wherein said antibody specifically recognizes the protein Trio and its isoforms chosen from TrioA, TrioC, TrioD and has a better sensitivity than the anti-Trio antibody H-120 (sc-28564, Santa Cruz Biotechnology. Inc) with respect to the presence of Trio or its isoforms in a biological sample, in particular such that at the concentration of 0.4 μg/ml, said antibody recognizes an amount below 100 pg, 80 pg, 70 pg, especially 50 pg of the Trio protein.

The specificity or sensitivity of the antibody can be determined by several means. An antibody is considered specific to target molecule when said antibody stains by western blot analysis a single band (or a set of bands) of the appropriate molecular mass for that target and that said antibody does not stain any band after knock down of the target by siRNA or in samples from trio k.o. mouse. The specificity can also be determined by immunoprecipitation. An antibody is considered to be specific to target molecule when said antibody immunoprecipitates a single band of appropriate molecular mass. Finally, the specificity of said antibody can be determined by immunocytochemistry or immunohistochemistry, using the same samples as before (comparison between normal cells and Trio-depleted cells).

An antibody is more sensitive than a reference antibody when it detects 2 to 10000 times less antigen than said reference antibody, for example the anti-Trio antibody H-120, at the same concentration. Alternatively, an antibody is more sensitive than a reference antibody when it can be used at a concentration from 2 to 10 times more diluted than said reference antibody to detect the same amount of antigen.

In the frame of the present invention, an anti-Trio antibody of the present invention stains only protein Trio having 340 kDa, and its isoform Trio A having 254 kDa, Trio C having 203 kDa, or Trio D having 303 kDa

The sensitivity of an antibody of the present invention can be determined by western-blot analysis or by flow cytometry analysis using the recombinant Trio protein as an antigen.

The antibody of the present invention has a sensitivity that is approximately 10-fold higher than the commercially available anti-Trio antibodies, such as the anti-Trio antibody H-120 (sc-28564) of Santa Cruz biotechnology, Inc.

In a particular embodiment, the antibody of the present invention is a polyclonal antibody enabled by an aforementioned antigen.

The term “Polyclonal antibody” refers to antibodies that are obtained from different B cell resources. They are a combination of immunoglobulin molecules secreted against a specific antigen, each identifying a different epitope.

In a more particular embodiment, the antibody of the present invention is a polyclonal antibody enabled by the antigen represented by SEQ ID NO: 4. Said polyclonal antibody is also designed below as TrioMTP.

Said polyclonal antibody recognizes Trio protein, and also isoform Trio A, Trio C and Trio D and the antigen represented by SEQ ID NO: 4.

The lower limit of detection for TrioMTP by western blot analysis is about 50 pg of the recombinant Trio protein.

Said polyclonal antibody can be produced by any conventional method, especially by a method comprising:

(i) inoculating an no-human mammal with an aforementioned antigen;

(ii) purifying the antibody from the serum of no-human mammal.

Such no-human mammal used in polyclonal antibody production can be rabbits, mouse, or goats.

The antigen of the present invention can be mixed with an immunological adjuvant, such as Freund's adjuvant, to stimulate immune response.

In another embodiment, the antibody of the present invention is a monoclonal antibody.

“Monoclonal antibody” refers to antibodies which are issued from a same B cell and recognize the same epitope of an antigen.

Said monoclonal antibody can be produced by any conventional method, especially by hybridoma obtained from the fusion of a B cell of immunized non-human animal and a myeloma cell.

In another embodiment, the present invention relates to a fragment of an above defined antibody, said fragment conserving the same or similar antigen binding capacity to that of said antibody.

The fragment according the invention can be SdAb, ScFv, Fab or F(ab′)₂ fragment.

SdAb, ScFv, Fab or F(ab′)₂ fragment used in the present invention can be produced by any method described in the prior art.

In a more particular embodiment, the present invention relates to a SdAb, ScFv, Fab or F(ab′)₂ fragment of an antibody raised against by the antigen represented by SEQ ID NO: 4.

An antibody or a fragment of an antibody of the present invention can be directly or indirectly labeled by a fluorophore.

By directly labeled antibody or antibody fragment, it is meant that said antibody or antibody fragment is directly linked to a fluorophore.

By indirectly labeled antibody or antibody fragment, it is meant that antibody or antibody fragment of the present invention can be recognized by a labeled molecule, for example a second labeled antibody which can recognize the antibody or antibody fragment of the present invention.

In a preferred embodiment, an antibody of the present invention is labeled by a fluorophore.

In another preferred embodiment, an antibody of the present invention is not labeled and can be recognized by a secondary antibody labeled by a fluorophore.

A fluorophore used for dying a primary or secondary antibody can be cyanine and its derivatives, such as Cy3 or Cy5, naphthalene and its derivatives, cumarin and its derivatives, rhodamine, xanthene, or commercial dyes, such as the Alexa Fluor family.

Such directly or indirectly labeled anti-Trio antibody can be used in immunoprecipitation, immunohistochemistry or immunocytochemistry.

The present invention also relates to a pharmaceutical composition comprising as active ingredient, an above defined antibody or fragment of antibody.

Said composition can comprise or consist in also a pharmaceutically acceptable vehicle.

In a preferred embodiment, a pharmaceutical composition of the present invention comprises or consists in an anti-Trio antibody raised against by the antigen represented by SEQ ID NO: 4.

Particularly, a pharmaceutical composition of the present invention can be used for cancer diagnostic, especially for diagnosing or prognosticating breast cancer, glioblastoma, bladder cancer, soft tissue sarcoma, uterus cancer, or lung cancer.

Especially, a pharmaceutical composition of the present invention can be used for diagnosing or prognosticating triple negative phenotype of breast cancer or soft tissue sarcoma with complex genetic profile.

The present invention also relates to an in vitro cancer prognostic method.

The method described in the present invention is based on the determination of at least one oncogenic marker expression level, including Trio protein.

In one embodiment, the prognostic method of the present invention comprises or consists in the determination of Trio protein expression level, with the help of an antibody defined in the present invention, in a tumoral sample.

In a particular embodiment, said method comprises or consists in:
(i) determining Trio protein expression level, with an antibody defined in the present invention, in a tumoral sample,
(ii) comparing Trio protein expression level measured in said sample with Trio protein expression level measured in a control sample from normal tissue or from another cancer sub-type

The term “cancer prognosis” refers to the prediction of the clinical outcome such as local recurrence or metastasis occurrence during a certain period of time (usually 5 to 10 years after the diagnosis) of a patient suffering from a diagnosed or detected cancer. The term may also refer to the survival probability of a patient suffering from a diagnosed or detected cancer.

Particularly, cancer prognosis can be defined by the prognostic index, such as association of the overexpression of a specific marker (Trio) with local recurrence or with distant metastasis-free survival (DMFS) after five or ten years after the cancer diagnosis.

The tumoral sample used in the present method is a tissue sample separated from the normal tissue of a patient suffering from a diagnosed or detected cancer, in particular breast cancer, glioblastoma, bladder cancer, soft tissue sarcoma, uterus cancer, or lung cancer. Tissue samples may be fresh, frozen or fixed paraffin embedded samples. In a particular embodiment, the tumoral sample may be a biopsy or resection sample.

The control used in the present invention is a sample from a healthy human or from another cancer sub-type. For example a sample of soft tissue sarcoma with amplification of chromosome 5p can be compared with another sample of soft tissue sarcoma without chromosome 5p amplification, which does not overexpress the Trio protein.

In a particular embodiment, the control may be a predetermined value, such as an average value of the values determined in a group of healthy humans.

The expression level of the Trio protein can be determined by immunochemical analysis by contacting the tumoral sample with an antibody or a fragment of an antibody defined in the present invention.

Particularly, the prognostic method of the present invention use an antibody evoked by the antigen represented by SEQ ID NO: 4, or a fragment, such as SdAb, ScFv, Fab or F(ab′)₂ fragment of the antibody evoked by the antigen represented by SEQ ID NO: 4.

According to the method of the present invention, the level of Trio expression in the control and the tumoral samples is compared. When Trio expression level is higher in the tumoral sample than in the control, the probability of relapse is more important. The probability of relapse associated with Trio overexpression in univariate analysis (local recurrence or distant metastasis-free survival (DMFS)) is calculated using the Kaplan-Meier method.

In an embodiment, the method described in the present invention is particularly for prognosticating breast cancer, glioblastoma, bladder cancer, soft tissue sarcoma, uterine cancer, or lung cancer.

In a particular embodiment, the present invention especially relates to a method for prognosticating triple negative breast cancer.

Triple negative phenotype of breast cancer refers to any breast cancer that does not express the genes for estrogen receptor (ER), progesterone receptor (PR) and Her2 (Perou et al. (2000) Nature, 406:747-752).

In a particular embodiment, the prognostic method of the present invention comprises or consists in the determination of the expression level of Trio with an anti-Trio antibody defined in the present invention, and of the expression level of at least one protein chosen from estrogen receptor, progesterone receptor, KI67, androgen receptors, Her2, CK5-6/EGFR/Vim, ALDH1, CD24, CD44, E-cadherin, BCL2, in a breast tumoral tissue sample.

In a more particular embodiment, the prognostic method of the present invention comprises or consists in the determination of the expression level of Trio with an antibody defined in the present invention, and the expression level of estrogen receptor (ER), progesterone receptor (PR) and Her2, in a breast tumoral tissue sample.

Particularly, said method comprises or consists in:
(i) determining the expression level of Trio, estrogen receptor (ER), progesterone receptor (PR) and Her2, in a breast tumoral tissue sample, wherein Trio protein expression level is determined with an antibody defined in the present invention
(ii) comparing Trio, ER, PR and Her2 protein expression levels measured in said sample with Trio, ER, PR and Her2 protein expression levels measured in a control.

When ER, PR and Her2 are not expressed in said tumoral sample and when Trio expression level in said tumoral sample is higher than in the control, there is a higher probability that said patient has poorer prognostic index, such as poorer distant metastasis-free survival (DMFS) over five or ten years.

In a more particular embodiment, the prognostic method of the present invention comprises or consists in the determination of the expression level of Trio with an antibody defined in the present invention, the expression level of estrogen receptor (ER), progesterone receptor (PR) and Her2, and the expression level of at least one protein chosen from CK5/6, vimentin, and EGFR, in a breast tumoral tissue sample.

Particularly, said method comprises or consists in:
(i) determining the expression level of Trio, estrogen receptor (ER), progesterone receptor (PR), Her2, and at least one protein chosen from CK5/6, vimentin, and EGFR, in a breast tumoral tissue sample, wherein Trio protein expression level is determined with an antibody defined in the present invention,
(ii) comparing the protein levels measured in said sample in step (i) with corresponding protein expression levels measured in a control.

In another particular embodiment, the present invention especially relates to a method for prognosticating soft tissue sarcoma.

Said method comprises or consists in the determination of the expression level of Trio with an anti-Trio antibody defined in the present invention in a soft tissue sarcoma tumoral sample with complex genetic profile.

Particularly, said method comprises or consists in:
(iii) determining Trio protein expression level, with an anti-Trio antibody defined in the present invention, in a soft sarcoma tumoral sample,
(iv) comparing Trio protein expression level measured in said sample with Trio protein expression level measured in a control.

When Trio expression level in said tumoral sample is higher than in the control, there is a greater probability that said patient has poorer prognostic index, such as local recurrence over five years.

Trio protein expression level can be determined by any conventional immunohistochemical method with an antibody defined in the present invention.

The present invention is further illustrated by following figures and examples.

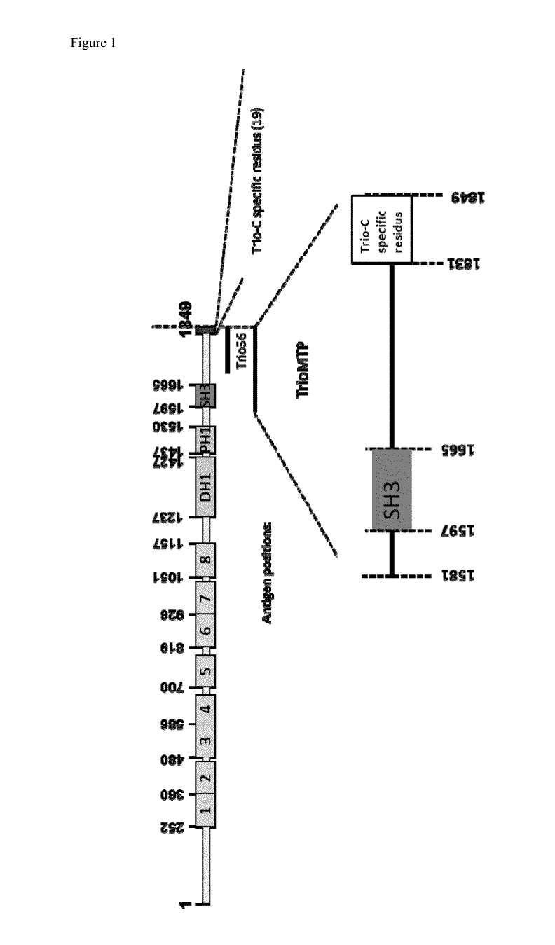

FIG. 1A illustrates the Trio-C protein, which has 8 spectrin repeats (1 to 8), a GEFD1 domain (DH1-PH1), a SH3 domain and a Trio-C specific C-terminal extremity (19 residues).

FIG. 1B illustrates the antigen of SEQ ID NO: 4 used to produce the antibody TrioMTP (1581aa to 1849aa), which contains the SH3 domain (1597aa to 1665aa) and the Trio-C specific C-terminal extremity (1831aa to 1849aa).

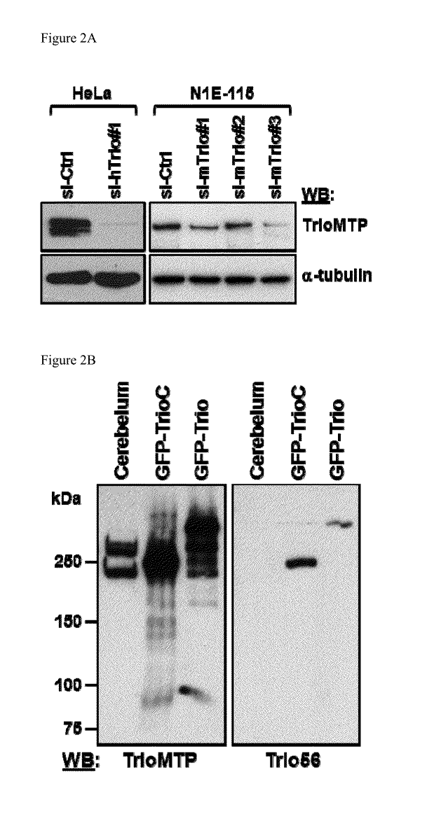

FIGS. 2A and 2B depict the detection of endogenous and recombinant Trio by Western immunoblotting with antibody TrioMTP raised against the antigen encompassing residues 1581-1849 of the Trio-C isoform (antigen of SEQ ID NO: 4).

FIG. 2A shows detection of endogenous Trio in HeLa and N1E-115 cells, which were transfected with either a control siRNA oligonucleotide or siRNA oligonucleotides targeting human (si-hTrio#1: GAUAAGAGGUACAGAGAUUUU) and mouse Trio (si-mTrio#1: GGAAGAAGUCUGAGAAGAA; si-mTrio#2: GCACAGAAGAUGAUGGAU; si-mTrio#3: GUACAAGCCAAGACGGAAAUU), respectively.

FIG. 2B shows western immunoblotting of a mouse cerebellar extract and lysates of HEK-293 cells expressing either pEGFP-Trio-C or pEGFP-Trio with antibody TrioMTP (left) and another anti-Trio-C antibody named Trio56 (right) raised against an antigen encompassing residues 1709-1849 of the Trio-C isoform.

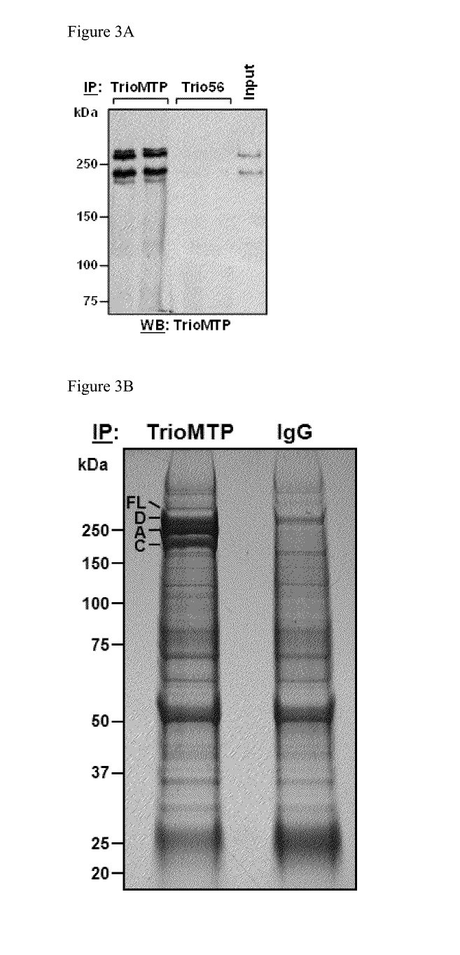

FIG. 3A shows the immunoprecipitation of endogenous Trio isoforms from 1 mg of mouse cerebellar extract by the antibodies TrioMTP and Trio56. The immunoprecipitates were electrophoresed on a polyacrylamide gel and were immunoblotted with the antibody TrioMTP. The lane “input” has been loaded with 20 μg of cerebellar extract.

FIG. 3B shows the immunoprecipitation of endogenous Trio isoforms from 100 mg of mouse cerebellar extract by the antibody TrioMTP and by control preimmune immunoglobulins (IgG). The immunoprecipitates were electrophoresed on a polyacrylamide gel and were visualized by colloidal blue Coomassie staining.

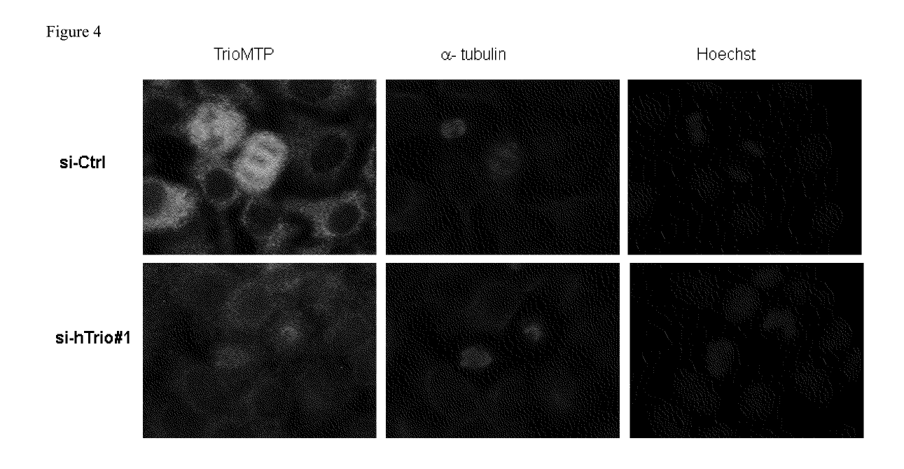

FIG. 4 shows endogenous Trio expression in HeLa cells detected by immunocytochemistry with the antibody TrioMTP. HeLa cells were transfected with a control siRNA oligonucleotide or a siRNA oligonucleotide targeting human Trio (si-hTrio#1). The cells were fixed 48 hours after transfection and labelled using the antibody TrioMTP. Tubulin was visualized using an antibody against α-tubulin, and nuclei were visualized using Hoechst staining.

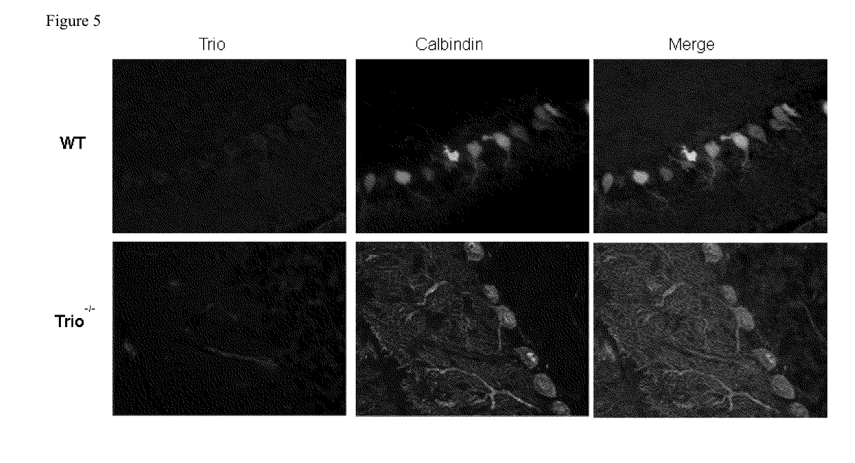

FIG. 5 shows endogenous Trio expression in mouse cerebellar slices detected by immunohistochemistry with the antibody TrioMTP. Conditional knockout mice whose cerebellar Purkinje cells lack Trio expression were generated by crossing trio flox/flox mice with a transgenic mice expressing the CRE recombinase under the pcp2 promoter (pcp2-CRE). Cerebellar cryosections from wild-type and conditional knockout Trio−/− mice were fixed and labelled using the antibody TrioMTP. The control labeling of Purkinje cells is carried out with an antibody against Calbindin, which is a Purkinje cell-specific marker.

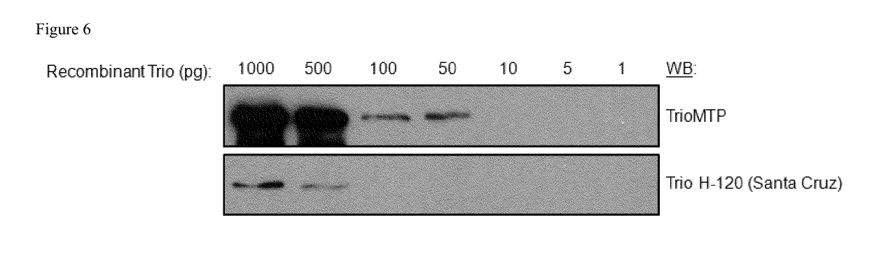

FIG. 6 shows the detection of the recombinant Trio protein by western blot using the antibody TrioMTP or a commercial anti-Trio antibody Trio H-120 (sc-28564). The indicated amounts of recombinant Trio purified from HEK-293 cells were electrophoresed in a polyacrylamide gel and immunoblotted with the indicated antibodies used at a concentration of 0.2 pg/ml.

### EXAMPLES

### 1. Materials and Methods

Immunisation Protocol for the Generation of the Antibody TrioMTP

Adult New Zealand White rabbits were used for antibody production. The antigen protein was diluted in PBS to a final concentration of 0.25 mg/ml. For initial inoculation (day 1), 400 μl (100 μg) of the antigen protein solution was mixed with 400 μl of complete Freund's adjuvent, and the antigen/adjuvent solution was inoculated subcutaneously. The rabbits received 3 boost injections of 100 μg of antigen mixed with incomplete Freund's adjuvent at days 7, 28 and 56. Blood (10 ml) was collected from the medial ear artery from day 35 at a frequency of one bleed per week for 18 weeks. For serum preparation, the whole blood was allowed to set at room temperature for 1 hour and then placed at 4° C. overnight. The following day, the clot was removed from the serum by centrifugation at 5,000 g for 10 min.

Production of the Antigen for the Generation of the Antibody TrioMTP

The fragment encompassing residues 1581-1849 of the Trio-C isoform was fused to a GST tag at the N-Terminus and expressed in E. coli. The pGEX-4T construct encoding the GST-tagged fragment was transformed into E. coli BL21 (DE3) cells and a 1-liter culture was grown at 37° C. in Luria-Bertani broth until the absorbance at 600 nm was ˜0.7. Isopropyl-β-D-thiogalactopyranoside (IPTG) was then added to a concentration of 250 μM to induce protein expression, and the cells were cultured for a further 16 h at 26° C. The cells were harvested by centrifugation at 5,000 g for 30 min and resuspended in 25 ml of a lysis buffer containing 50 mM Tris-HCl pH 7.5, 150 mM NaCl, 1% (w/v) Triton X-100, 1 mM DTT, 1 mM benzamidin, 0.1 mM phenylmethylsulfonyl (PMSF), and 0.2 mg/ml lysozyme. Lysis was completed by one round of freeze/thawing and ten 15-sec cycles of sonication. The lysate was centrifuged at 20,000 g for 30 min at 4° C., and the supernatant was incubated for 1 h on a rotating platform with 0.5 ml of glutathione-Sepharose previously equilibrated in lysis buffer. The beads were washed 4 times with 50 mM HEPES pH 7.4, 500 mM NaCl, 1 mM DTT, and twice with 50 mM HEPES pH 7.4, 1 mM DTT. The proteins were eluted from the resin by incubation for 30 min with the same buffer containing 20 mM of reduced glutathione pH 8.0. The beads were then removed by filtration through a 0.44-μm filter.

Preparation of the Affinity Columns and Purification of the Antibodies

The eluted antigen proteins (0.5 mg) were covalently coupled to Affi-Gel sepharose (0.5 ml of packed resin; BioRad) using the manufacturer's procedure. For affinity purification of antibodies, each serum was filtered through a 0.44-μm filter and diluted twice in PBS. The diluted serum (10 ml) was passed 3 times through the affinity column coupled to the corresponding antigen protein, which was then washed 3 times with PBS. The antibodies retained onto the affinity column were eluted with 0.8 ml of 0.1 M Glycine pH 2.5 and neutralised by the addition of 0.2 ml of 1 M Tris-HCl pH 8.0. The resulting antibody preparation was then passed through an Affi-Gel sepharose column coupled to the GST protein in order to retain the anti-GST antibodies contained in the preparation.

Mouse Cerebellar Extract Preparation

Mouse (P27) cerebella were disrupted and homogenized in a Dounce homogenizer in lysis buffer using 20 strokes. The resulting extract was clarified by centrifugation at 20,000 g for 10 minutes at 4° C.

Western Immunoblotting

Protein extracts were resolved by SDS-PAGE and transferred to nitrocellulose membranes. The membranes were incubated in TBS containing 0.5% Tween-20 (TBST) and 10% (w/v) skimmed milk powder, and then incubated in the same buffer with 0.2 μg/ml of the indicated primary antibodies. The membranes were then washed six times with TBST and incubated with secondary HRP-conjugated antibodies in TBST containing 10% (w/v) skimmed milk powder. After repeating the washing steps, the signal was detected with the enhanced chemiluminescence (ECL) reagent.

Cell Culture and Transfection

HEK-293, HeLa and neuroblastoma N1E-115 cells were cultured in 100-mm dishes in Dulbecco's Modified Eagle's Medium (DMEM) supplemented with 10% (v/v) fetal bovine serum, 2 mM L-glutamine, 50 U/ml penicillin and 50 μg/ml streptomycin at 37° C. with 5% CO₂. For fluorescence microscopy experiments, HeLa and N1E-115 cells were plated in 35-mm dishes (1.25×10⁶ cells/plate) containing glass coverslips. For N1E-115 cells, the glass coverslips were coated with laminin (25 μg/ml, BD Biosciences). Cells were transfected with the indicated constructs using linear polyethylenimine (PEI, PolySciences) according to the manufacturer's instructions. Cells were lysed in lysis buffer containing 50 mM Tris-HCl pH 7.5, 1% (w/v) Triton X-100, 1 mM EGTA, 1 mM EDTA, 50 mM sodium fluoride, 5 mM sodium pyrophosphate, 1 mM sodium orthovanadate, 0.27 M sucrose, 1 mM DTT, 1 mM benzamidin, and 0.1 mM phenylmethylsulfonyl (PMSF). Cells lysates were clarified by centrifugation at 20,000 g for 10 minutes at 4° C., and protein concentrations were determined using the Bradford method.

siRNA Knock Down

Synthetic siRNA oligonucleotides were generated by Dharmacon RNAi Technologies (Thermo Scientific). The sequence of the oligonucleotide targeting human Trio was GAUAAGAGGUACAGAGAUUUU (si-hTrio#1). The sequences of the oligonucleotides targeting mouse Trio were GGAAGAAGUCUGAGAAGAA (si-mTrio#1), GCACAGAAGAUGAUGGAU (si-mTrio#2) and GUACAAGCCAAGACGGAAAUU (si-mTrio#3). The control siRNA oligonucleotide was the siGENOME Non-Targeting siRNA#1 from Dharmacon RNAi Technologies. Cells were transfected with 200 nM of the indicated siRNA oligonucleotides were either lysed or fixed 48 hours after transfection.

Immunoprecipitation of Trio

For large-scale immunoprecipitation of Trio, 100 mg of clarified mouse cerebellar extract was preincubated with 0.1 mg preimmune antibody covalently conjugated to 0.1 ml protein G-Sepharose for 2 hours at 4° C. After centrifugation, the supernatant was incubated with 0.1 mg anti-TrioMTP antibody covalently conjugated to 0.1 ml protein G-Sepharose for 2 h at 4° C. The immunoprecipitates were washed three times with lysis buffer containing 0.15 M NaCl but no reducing agent, and twice with 10 mM Tris-HCl pH7.5, 0.1 mM EDTA. The beads were resuspended in SDS sample buffer and the samples were heated to 100° C. for 10 minutes. The eluted proteins were subjected to electrophoresis on a 4-12% SDS-polyacrylamide gel (Invitrogen), which was stained with colloidal blue Coomassie. The small-scale immunoprecipitation of Trio was performed as above except that 1 mg of cerebellar extract was used with 5 μg of the TrioMTP antibody, and that the immunoprecipitates were immunoblotted with the same antibody.

Immunocytochemistry

HeLa cells grown on glass coverslips and transfected with the indicated siRNA oligonucleotides were fixed in 100% methanol for 5 minutes at −20° C. and then rehydrated in Dulbecco's PBS for 30 minutes. The fixed cells were blocked in PBS containing 3% BSA for 5 minutes at room temperature and then incubated with the rabbit anti-TrioMTP (1:100) or the mouse anti-a-tubulin (BD Biosciences; 1:200) primary antibodies diluted in PBS-3% BSA for 1 hour at room temperature. After washing in PBS, the coverslips were incubated with Alexa Fluor 488-conjugated goat anti-rabbit (1:2000) or rhodamine-conjugated goat anti-mouse (1:100) secondary antibodies (Life Technologies) respectively, diluted in PBS-3% BSA, together with 1 mg/ml Hoechst dye, for 45 minutes at room temperature. After washing in PBS, the coverslips were mounted using Mowiol medium. The slides were visualised with a Leica widefield DM6000 microscope using a 63×1.4 N.A. oil objective, and images were acquired with a CCD HQ2 devices camera (Roper Scientific) controlled by Metamorph 7.1 software (Universal Imaging).

Immunohistochemistry

Immunohistochemistry on TMA (tumor micro array) was performed concurrently on serial sections. Slides were treated on Benchmark®XT (Ventana).

Rabbit polyclonal anti-Trio antibody (Trio-MTP) was applied at 0.3 mg/ml (1/100-HIER pH9) over night at 4° C. After treatment with the primary antibodies, the slides were washed in TBST (Dako, wash buffer) and detected with horseradish peroxidase-conjugated anti-mouse/rabbit Envision+ kit (Dako). All slides were counterstained with haematoxylin.

Immunohistochemistry on mouse cerebellar slices were performed as follows. Mice were perfused transcardially with 4% paraformaldehyde (PFA) in 0.12 M phosphate buffer pH 7.4. Brains were isolated, post-fixed overnight in 4% PFA, cryoprotected in 20% then 30% sucrose, embedded in OCT, and frozen in isopentane at −55° C. and stored at −80° C. until sectioning. Sagittal sections (10 mm) were cut with a cryostat and rehydrated in PBS for 5 minutes, then fixed and permeabilised in 70% methanol-30% acetone for 15 minutes at −20° C. After washing in PBS, the slices were blocked in PBS containing 1% BSA for 30 minutes at room temperature and then incubated with the rabbit anti-TrioMTP (1:500) or the mouse anti-calbindin (Swant; 1:5000) primary antibodies diluted in PBS-1% BSA for 2 hours at room temperature. After washing in PBS, the slices were incubated with rhodamine-conjugated goat anti-rabbit (1:1000) or Alexa Fluor 488-conjugated goat anti-mouse (1:2000) secondary antibodies (Life Technologies) respectively, diluted in PBS-1% BSA for 45 minutes at room temperature. After washing in PBS, the slices were mounted using Mowiol medium. The slides were visualised with a Leica widefield DM6000 microscope using a 40×1.25 N.A. oil objective, and images were acquired as above.

Production of Trio Conditional Knockout Mice

Trio conditional knockout mice (trio flox/flox) were generated by the ICS (Institut Clinique de la Souris, Strasbourg, http://www.ics-mci.fr). To specifically delete Trio in Purkinje cells, trio flox/flox mice were crossed with a transgenic mice expressing the CRE recombinase under the pcp2 promoter, which is specific for Purkinje cells after birth. (Rico, B. et al., Nat Neurosci, 2004. 7 (10): p. 1059-69.)

### 2. Results

Detection of Endogenous and Recombinant Trio by Western Immunoblotting

Endogenous and recombinant Trio are detected by western immunoblotting with the TrioMTP antibody of the present invention raised against the antigen containing the SH3 domain and the Trio56 antibody raised against an antigen without the SH3 domain.

The polyclonal TrioMTP and Trio56 antibodies were raised against fragments encompassing residues 1581-1849 and 1704-1849 of the Trio-C isoform, respectively.

The TrioMTP antibody specifically recognizes the endogenous protein Trio, since the signal is reduced when Trio is depleted in cells by siRNA knock down (FIG. 2A).

The TrioMTP antibody is also much more sensitive than the Trio56 antibody, and recognizes all Trio isoforms whereas the Trio56 antibody recognizes only Trio-C and weakly Trio (FIG. 2B).

The TrioMTP antibody is also much more sensitive than the H-120 antibody (Santa Cruz biotechnology, Inc). The lower limit of detection for TrioMTP is 50 pg of Trio compared to 500 pg for Trio H-120, showing that the TrioMTP antibody has a 10-fold higher sensitivity than the H-120 antibody (FIG. 6).

Immunoprecipitation of Endogenous Trio from Mouse Cerebellum

The endogenous Trio isoforms were immunoprecipitated from 1 mg (FIG. 3A) or 100 mg (FIG. 3B) of mouse cerebellar extract using the TrioMTP or the Trio56 antibodies.

The results of FIGS. 3A and 3B show that the TrioMTP antibody is very efficient in immunoprecipitating all Trio isoforms from mouse cerebellar extract whereas Trio56 is unable to do so.

Detection of Endogenous Trio by Immunocytochemistry

Endogenous Trio was detected by immunocytochemistry in HeLa cells transfected with either a siRNA control or a siRNA targeting human Trio, using the TrioMTP antibody.

This antibody specifically recognizes Trio, as the signal observed is reduced when Trio expression is depleted in cells (FIG. 4).

Detection of Endogenous Trio by Immunohistochemistry

Endogenous Trio was detected by immunohistochemistry in Purkinje cells of cerebellar slices from wild-type and conditional knockout mice lacking Trio expression with the TrioMTP antibody.

This antibody specifically detects Trio by immunohistochemistry since the signal is absent when Trio is not expressed in the tissue (FIG. 5).

Correlation Between Trio Expression and Triple Negative Breast Cancer

Between 1989 and 1993, 1634 patients with primary operable invasive breast carcinoma were operated on and monitored at the “Institut Bergonie” (Bordeaux). Tissue microarray (TMA) blocks were built for immunohistochemical analyses and 1124 cases available for the study (1018 invasive ductal carcinoma not otherwise specified (IDC-NOS) and 106 invasive lobular carcinoma (ILC)). Median follow-up was 13.7 years [95% CI (13.6-14)]. Patients were treated with surgery, by either modified radical mastectomy (270 cases) or local tumor resection (854 cases), with axillary node dissection in 1101 cases (98%). Post-operative breast irradiation was performed in 879 cases. Adjuvant therapy with chemotherapy and/or hormone therapy was decided according to nodal status and hormone receptor determination results. All patients were followed in the cancer center “Institut Bergonié”.

The expression levels of Trio, estrogen receptor, progesterone receptor, KI67, androgen receptors, Her2, CK5-6/EGFR/Vim, ALDH1, CD24, CD44, E-cadherin, BCL2, were determined by immunohistochemistry.

Among the 1124 breast tumors, there were 115 (10%) triple negative tumors (TN). The expression levels of ER, PR, HER2 and Trio protein are illustrated in the Table 1. Tumors were considered as positive for estrogen (ER) and progesterone (PR) receptors when ≧10% of cells were positive for expression and for HER2 if the Herceptest showed 3+ staining intensity. Tumors with more than 60% of 2+ positive tumor cells were considered as HER2 positive (Chibon et al., (2009) Mod. Pathol. 22: 403-409). Cases were categorized as basal immunophenotype when at least one out of the three markers (CK5/6, EGFR, and vimentin) was positive. Expression of Trio and other markers was considered as positive when 1% or more of cells were positive. There was no difference in expression of Trio according to molecular groups. In the TN tumor group, 50% of the tumors were Trio-positive (see table 1 below). The TN group was further defined into basal-like (ER/PR/HER2 negative, CK5/6 and/or vimentin and/or EGFR positive) or non-basal-like (ER/PR/HER2 negative, CK5/6, vimentin and EGFR negative). Trio expression was present in both sub-types, with more expression in the non-basal like subgroup (Table 2).

TABLE 1

| | Triple negative n = 115 (%) |
|---|---|
| Estrogen receptor | |
| 0% | 113 (98) |
| 1-9% | 2 (2) |
| Positive | — |
| Not specified | |
| Progesterone receptor | |
| Negative | 115 (100) |
| Positive | — |
| Not specified | — |
| HER2 | |
| Negative | 115 (100) |
| Positive | |
| Trio phenotype | |
| Negative | 65 (56) |
| Positive | 49 (43) |
| Not specified | 1 (1) |

TABLE 2

| | Non-basal-like TN | Basal-like TN |
|---|---|---|
| Trio phenotype | (n = 21) | (n = 94) |
| Negative | 7 (33) | 58 (62) |
| Positive | 14 (67) | 35 (38) |
| Not specified | — | 1 (1) |

Univariate and multivariate analyses were carried out for association between clinicopathological, microenviromental and immunohistochemical phenotypes and distant metastasis-free survival (DMFS) over the first five years in each molecular group.

Univariate and multivariate analyses were calculated using the Cox model for DMFS over the time period with no risk proportionality violations. Only variables significant (p <0.05) in the univariate were maintained in the multivariate models which used a stepwise ascending maximum likelihood method.

In univariate analyses for TN tumors, a worst DMFS was associated with the high expression of Trio, (HR=2.2 95% CI [1.1-4.3]), suggesting that patients expressing high level of Trio have an increased risk of developing metastases.

In multivariate analyses for TN tumors, a worst DMFS was also associated with the expression of Trio, (HR=2.8 95% CI [1-4.6]).

All together, these data suggest that the expression of Trio is an independent pejorative factor in breast TN tumors.

1. An antigen constituted of from 50 to 400 amino acids, said antigen comprising a polypeptide P1,
which is a peptide of at least 50 contiguous amino acids belonging to the polypeptide represented by sequence SEQ ID NO: 1;
with the proviso that
LSGGCELTVVIHDFTACNSNELTIRRGQTVEVLERPHDKPDWCLVRTTDR
SPAAEGLVPCGSLCIAHSRSSMEMEGIFNHK is excluded,
or
which is a peptide having at least 90%, in particular 95%, especially 98% of sequence homology with the amino acid sequence of SEQ ID NO: 1,
in particular the polypeptide P1 being represented by amino acid sequence SEQ ID NO: 1,
with the proviso that
LSGGCELTVVIHDFTACNSNELTIRRGQTVEVLERPHDKPDWCLVRTTDR
SPAAEGLVPCGSLCIAHSRSSMEMEGIFNHK is excluded.

2. An antigen according to claim 1, comprising:
a polypeptide P1, situated at N-terminal part of said antigen, having the amino acid sequence of SEQ ID NO: 1 or an amino acid sequence having at least 90%, in particular 95%, especially 98% of sequence homology with the sequence of SEQ ID NO: 1, and
a polypeptide P2, situated at C-terminal part of said antigen, having the amino acid sequence of SEQ ID NO: 2 or an amino acid sequence having at least 90%, in particular 95%, especially of 98% of sequence homology with the sequence of SEQ ID NO:2.

3. An antigen P3 according to claim 2, having at least from 100 to 300 amino acids between the C-terminal part of the polypeptide P1, having the amino acid sequence of SEQ ID NO: 1 or an amino acid sequence having at least 90%, in particular 95%, especially of 98% of sequence homology with the sequence of SEQ ID NO:1 and the N-terminal part of the polypeptide P2 having the amino acid sequence of SEQ ID NO: 2 or an amino acid sequence having at least 90%, in particular 95%, especially of 98% of sequence homology with the sequence of SEQ ID NO:2.

4. An antigen according to claim 3,
wherein the polypeptide P3 has the amino acid sequence of SEQ ID NO: 3 or an amino acid sequence having at least 90%, in particular 95%, more in particular 98% of sequence homology with SEQ ID NO: 3, or
wherein the polypeptide P3 is a fragment of from 50 to 165 contiguous amino acids of a peptide represented by SEQ ID NO: 3, or
wherein the N-terminal extremity of the polypeptide P3 is located at position 1 of the sequence SEQ ID: 3 and the C-terminal extremity of the polypeptide P3 is located from position 50 to position 165 of the sequence SEQ ID NO: 3.

5. An antigen according to claim 1, comprising an amino acid represented by the sequence of SEQ ID NO: 4 or an amino acid sequence having at least 90%, in particular 95%, more in particular 98% of sequence homology with SEQ ID NO:4.

6. An antigen according to claim 5, represented by the sequence of SEQ ID NO: 4.

7. A nucleic acid coding for an antigen such as defined in claim 1.

8. A nucleic acid according to claim 7, having the sequence SEQ ID NO: 7 coding for the amino acid represented by the sequence of SEQ ID NO: 4.

9. A recombinant vector, especially a plasmid, a cosmid, a phage or a DNA virus comprising a nucleic acid according to claim 7, eventually said recombinant vector containing the elements necessary for the expression in a host cell of the antigen coded by the nucleic acids according to claim 7, inserted in said vector.

10. A host cell, in particular chosen from bacteria, viruses, yeasts, fungi, plants or mammalian cells, the said host cell being transformed, especially by means of a vector, in such a way that a genome of the host cell contains a nucleotide sequence according to claim 7;
wherein the vector is a recombinant vector, especially a plasmid, a cosmid, a phage or a DNA virus comprising a nucleic acid according to claim 7, eventually said recombinant vector containing the elements necessary for the expression in a host cell of the antigen coded by the nucleic acids according to claim 7, inserted in said vector.

11. An antibody against the antigen according to claim 1, wherein said antibody specifically recognizes the Trio protein and its isoforms thereof chosen from TrioA, TrioC, TrioD and has a better sensitivity than the anti-Trio antibody H-120 (sc-28564, Santa Cruz Biotechnology. Inc) with respect to the presence of Trio or isoforms thereof in a biological sample, in particular such that at the concentration of 0.2 μg/ml, said antibody recognizes an amount below 100 pg, 80 pg, 70 pg, especially 50 pg of Trio protein by western blot analysis, in particular said antibody is a polyclonal antibody, or a monoclonal antibody, or a fragment of an antibody wherein said fragment is a SdAb, scFv, Fab or F(ab′)₂.

12. A pharmaceutical composition comprising or consisting in, as active ingredient, an antibody according to claim 11.

13. In vitro cancer prognostic method, comprising:
(i) determining Trio protein expression level, with the help of an antibody according to claim 11, in a tumoral sample,
(ii) comparing Trio protein expression level measured in said sample with Trio protein expression level measured in a control sample.

14. Method according to claim 13, wherein said cancer is chosen from breast cancer, in particular breast cancer of triple negative phenotype, glioblastoma, bladder cancer, soft tissue sarcoma, uterine cancer, or lung cancer.

15. An antigen constituted of from 50 to 400 amino acids, said antigen comprising a polypeptide P1,
which is a peptide of at least 50 contiguous amino acids belonging to the polypeptide represented by sequence SEQ ID NO: 1;
or
which is a peptide having at least 90%, in particular 95%, especially 98% of sequence homology with the amino acid sequence of SEQ ID NO: 1,
in particular the polypeptide P1 being represented by amino acid sequence SEQ ID NO: 1, or an antigen according to claim 1, for use in the treatment or the prophylaxis of cancer.

16. An antigen according to claim 2, comprising an amino acid represented by the sequence of SEQ ID NO: 4 or an amino acid sequence having at least 90%, in particular 95%, more in particular 98% of sequence homology with SEQ ID NO:4.

17. An antigen according to claim 3, comprising an amino acid represented by the sequence of SEQ ID NO: 4 or an amino acid sequence having at least 90%, in particular 95%, more in particular 98% of sequence homology with SEQ ID NO:4

18. An antigen according to claim 4, comprising an amino acid represented by the sequence of SEQ ID NO: 4 or an amino acid sequence having at least 90%, in particular 95%, more in particular 98% of sequence homology with SEQ ID NO:4

19. A nucleic acid coding for an antigen such as defined in claim 2.

20. A nucleic acid coding for an antigen such as defined in claim 3.


SEQUENCE LISTING (10 sequences)

SEQ ID NO: 1 - protein, 69 aa, Homo sapiens
GGCELTVVIHDFTACNSNELTIRRGQTVEVLERPHDKPDWCLVRTTDRSPAAEGLVPCGSLCIAHSRSS

SEQ ID NO: 2 - protein, 19 aa, Homo sapiens
HYVDLCSVSVLAQFPYLSV

SEQ ID NO: 3 - protein, 165 aa, Homo sapiens
MEMEGIFNHKDSLSVSSNDASPPASVASLQPHMIGAQSSPGPKRPGNTLRKWLTSPVRRLSSGKADGHVKKLAHKHKKSREVRKSADAGSQKDSDDSAATPQDETVEERGRNEGLSSGTLSKSSSSGMQSCGEEEGEEGADAVPLPPPMAIQQHSLLQPDSQDDK

SEQ ID NO: 4 - protein, 269 aa, Homo sapiens
IASRTSQNTLDSDKLSGGCELTVVIHDFTACNSNELTIRRGQTVEVLERPHDKPDWCLVRTTDRSPAAEGLVPCGSLCIAHSRSSMEMEGIFNHKDSLSVSSNDASPPASVASLQPHMIGAQSSPGPKRPGNTLRKWLTSPVRRLSSGKADGHVKKLAHKHKKSREVRKSADAGSQKDSDDSAATPQDETVEERGRNEGLSSGTLSKSSSSGMQSCGEEEGEEGADAVPLPPPMAIQQHSLLQPDSQDDKHYVDLCSVSVLAQFPYLSV

SEQ ID NO: 5 - DNA, 207 bp, Homo sapiens
ggtggctgtgagctgacagtggtgatccatgacttcaccgcttgcaacagcaacgagctgaccatccgacggggccagaccgtggaagttctggagcggccgcatgacaagcctgactggtgtctggtgcggaccactgaccgctccccagcggcagaaggcctggtcccctgtggttcactgtgcatcgcccactccagaagtagc

SEQ ID NO: 6 - DNA, 60 bp, Homo sapiens
cattatgttgatttgtgttctgtgtctgttttggctcaatttccttatctttccgtttga

SEQ ID NO: 7 - DNA, 810 bp, Homo sapiens
atcgcctcacggacgtctcagaacacgctggacagcgataagctctctggtggctgtgagctgacagtggtgatccatgacttcaccgcttgcaacagcaacgagctgaccatccgacggggccagaccgtggaagttctggagcggccgcatgacaagcctgactggtgtctggtgcggaccactgaccgctccccagcggcagaaggcctggtcccctgtggttcactgtgcatcgcccactccagaagtagcatggaaatggagggcatcttcaaccacaaagactcgctctccgtctccagcaatgacgccagtccacccgcatccgtggcttccctccagccccacatgatcggggcccagagctcgccgggccccaagcggccgggcaacaccctgcgcaagtggctcaccagccccgtgcggcggctcagcagcggcaaggccgacgggcacgtgaagaagctggcgcacaagcacaagaagagccgcgaggtccgcaagagcgccgacgccggctcgcagaaggactccgacgacagtgcggccaccccgcaggacgagacggtcgaggagagaggccggaacgagggcctgagcagcggtactctctccaaatcctcctcctcggggatgcagagctgtggagaagaggaaggcgaggagggggccgacgccgtgcccctgccgccacccatggccatccagcagcacagcctcctccagccagactcacaggatgacaagcattatgttgatttgtgttctgtgtctgttttggctcaatttccttatctttccgtttga

SEQ ID NO: 8 - protein, 1596 aa, Homo sapiense
MKAMDVLPILKEKVAYLSGGRDKRGGPILTFPARSNHDRIRQEDLRRLISYLACIPSEEVCKRGFTVIVDMRGSKWDSIKPLLKILQESFPCCIHVALIIKPDNFWQKQRTNFGSSKFEFETNMVSLEGLTKVVDPSQLTPEFDGCLEYNHEEWIEIRVAFEDYISNATHMLSRLEELQDILAKKELPQDLEGARNMIEEHSQLKKKVIKAPIEDLDLEGQKLLQRIQSSESFPKKNSGSGNADLQNLLPKVSTMLDRLHSTRQHLHQMWHVRKLKLDQCFQLRLFEQDAEKMFDWITHNKGLFLNSYTEIGTSHPHAMELQTQHNHFAMNCMNVYVNINRIMSVANRLVESGHYASQQIRQIASQLEQEWKAFAAALDERSTLLDMSSIFHQKAEKYMSNVDSWCKACGEVDLPSELQDLEDAIHHHQGIYEHITLAYSEVSQDGKSLLDKLQRPLTPGSSDSLTASANYSKAVHHVLDVIHEVLHHQRHVRTIWQHRKVRLHQRLQLCVFQQEVQQVLDWIENHGEAFLSKHTGVGKSLHRARALQKRHEDFEEVAQNTYTNADKLLEAAEQLAQTGECDPEEIYQAAHQLEDRIQDFVRRVEQRKILLDMSVSFHTHVKELWTWLEELQKELLDDVYAESVEAVQDLIKRFGQQQQTTLQVTVNVIKEGEDLIQQLRDSAISSNKTPHNSSINHIETVLQQLDEAQSQMEELFQERKIKLELFLHVRIFERDAIDIISDLESWNDELSQQMNDFDTEDLTIAEQRLQHHADKALTMNNLTFDVIHQGQDLLQYVNEVQASGVELLCDRDVDMATRVQDLLEFLHEKQQELDLAAEQHRKHLEQCVQLRHLQAEVKQVLGWIRNGESMLNAGLITASSLQEAEQLQREHEQFQHAIEKTHQSALQVQQKAEAMLQANHYDMDMIRDCAEKVASHWQQLMLKMEDRLKLVNASVAFYKTSEQVCSVLESLEQEYKREEDWCGGADKLGPNSETDHVTPMISKHLEQKEAFLKACTLARRNADVFLKYLHRNSVNMPGMVTHIKAPEQQVKNILNELFQRENRVLHYWTMRKRRLDQCQQYVVFERSAKQALEWIHDNGEFYLSTHTSTGSSIQHTQELLKEHEEFQITAKQTKERVKLLIQLADGFCEKGHAHAAEIKKCVTAVDKRYRDFSLRMEKYRTSLEKALGISSDSNKSSKSLQLDIIPASIPGSEVKLRDAAHELNEEKRKSARRKEFIMAELIQTEKAYVRDLRECMDTYLWEMTSGVEEIPPGIVNKELIIFGNMQEIYEFHNNIFLKELEKYEQLPEDVGHCFVTWADKFQMYVTYCKNKPDSTQLILEHAGSYFDEIQQRHGLANSISSYLIKPVQRITKYQLLLKELLTCCEEGKGEIKDGLEVMLSVPKRANDAMHLSMLEGFDENIESQGELILQESFQVWDPKTLIRKGRERHLFLFEMSLVFSKEVKDSSGRSKYLYKSKLFTSELGVTEHVEGDPCKFALWVGRTPTSDNKIVLKASSIENKQDWIKHIREVIQERTIHLKGALKEPIHIPKTAPATRQKGRRDGEDLDSQGDGSSQPDTISIASRTSQNTLDSDKLS

SEQ ID NO: 9 - protein, 16 aa, Homo sapiens
IASRTSQNTLDSDKLS

SEQ ID NO: 10 - DNA, 495 bp, Homo sapiens
atggaaatggagggcatcttcaaccacaaagactcgctctccgtctccagcaatgacgccagtccacccgcatccgtggcttccctccagccccacatgatcggggcccagagctcgccgggccccaagcggccgggcaacaccctgcgcaagtggctcaccagccccgtgcggcggctcagcagcggcaaggccgacgggcacgtgaagaagctggcgcacaagcacaagaagagccgcgaggtccgcaagagcgccgacgccggctcgcagaaggactccgacgacagtgcggccaccccgcaggacgagacggtcgaggagagaggccggaacgagggcctgagcagcggtactctctccaaatcctcctcctcggggatgcagagctgtggagaagaggaaggcgaggagggggccgacgccgtgcccctgccgccacccatggccatccagcagcacagcctcctccagccagactcacaggatgacaag